(19) United States
(12) Patent Application Publication
(10) Pub. No.: US 2022/0362412 A1
(43) Pub. Date: Nov. 17, 2022

(54) METHOD FOR DEODORIZING ALGAE

(71) Applicant: Nanoalgae Solutions AG, Hergiswil (CH)

(72) Inventors: Theocharis V. KOFTIS, Thessaloniki (GR); Efstratios NEOKOSMIDIS, Thessaloniki (GR); Michael TERZIDIS, Thessaloniki (GR); Theodoros PANAGIOTIDIS, Thessaloniki (GR); Thanos ANDREOU, Thessaloniki (GR); Elli VASTARDI, Thessaloniki (GR); Foteini ATHANASIADOU, Thessaloniki (GR)

(21) Appl. No.: 17/623,963

(22) PCT Filed: Jul. 1, 2020
(86) PCT No.: PCT/EP2020/025308

§ 371 Date: Dec. 30, 2021

(30) Foreign Application Priority Data
Jul. 2, 2019 (EP) PCT/EP2019/025206

(51) Int. Cl. A61L 2/00; C12N 1/12

(52) U.S. Cl. CPC A61L 2/0005; C12N 1/125

(57) ABSTRACT

A non-destructive and reversible process to deodorize Nannochloropsis algae is provided.

### TECHNICAL FIELD OF THE INVENTION

The present invention relates to the provision of a process for deodorizing algae. The invention particularly relates to a process of removing the odoriferous substances, especially the ones responsible for the fishy smell found in algae, from algae of the Nannochloropsis genus.

### BACKGROUND OF THE INVENTION

Algae are single-celled organisms that grow in water. They use photosynthesis to turn light, carbon dioxide and nutrients into oils, carbohydrates and proteins. Their cultivation does not require arable land and they can in fact be grown in brackish, fresh, or seawater at a fast growth rate.

Algae in the dry powder form, may be consumed neat, or may be used as a food additive in functional and healthy mass-market food, such as food and drink products., i.e. bread, dairy products, meat, fruit juices, nutrition bars, infant food, etc., long shelf-life microalgae-based nutraceuticals, such as tablets or plain powder for health supplementation, in personal care products and as fish feed ingredient in aquaculture.

Recent studies indicate that the global population is growing and along with this the food requirements are increasing, while at the same time resources and arable land are becoming progressively limited.

Microalgae can be induced to produce specific lipids and fatty acids through relatively simple manipulations of the physical and chemical properties of their culture medium. They can produce and accumulate substantial amounts of lipids, up to 20-50% of dry weight. The accumulation of lipids in microalgae is attributed to consumption of sugars at a rate higher than the rate of cell regeneration, which promotes the conversion of excess sugar into lipids. Polyunsaturated fatty acids (PUFAs) such as the omega-3 fatty acids are vital to everyday life and function. The beneficial effects of omega-3 fatty acids on lowering serum triglycerides are now well established. These compounds are also known for other cardioprotective benefits, such as cholesterol level reduction, protection against coronary heart disease and suppression of platelet aggregation. Other benefits of PUFAs are those related to the prevention and/or treatment of inflammation, neurodegenerative diseases, and cognitive development.

Nannochloropsis is one of the algae genera used to produce high value oil containing PUFAs, among which eicosapentaenoic acid (EPA) is of particular value and interest. Over two thirds of the fatty acids produced by Nannochloropsis consist of EPA, palmitic acid and palmitoleic acid.

Microalgae are a rich and sustainable source of plant protein, whose content varies across the many species. Arthrospira platensis (Spirulina) and Chlorella are the most widely used species commercially in this category, and may contain up to 70% wt protein. Protein derived from algae may be consumed as a nutritional supplement in the form of tablets or powder, or as additives in foods such as pasta, bread, dairy, nutrition bars for sports nutrition, etc.

Microalgae produce a wide range of volatile compounds which are often responsible for the unpleasant flavor and/or odor. PUFAs present in algae, are very sensitive to oxidation due to the high degree of unsaturation, which results in the decomposition of PUFAs and formation of primary and secondary oxidation products which are known to have an unpleasant taste and/or odor. This may be attributed to the formation of aldehydes, ketones and alcohols. In many of the algae applications mentioned above, in dairy products for instance, it is desirable to eliminate this taste and smell, at the highest degree possible, and provide a product which is not only of a superior nutritional value, but at the same time attractive to the consumer.

Methods to deodorize algae or algae derived products in the art, are, in most cases, applied to the oils deriving therefrom. Vacuum steam distillation at high temperatures and further optional steps such as contacting the oil with adsorbents such as silica, addition of antioxidants such as ascorbyl palmitate, tocopherol and lecithin are commonly used methods to deodorize algal, and generally marine oils. In some cases rosemary or sage extracts are used as antioxidants, occasionally altering odor and taste.

Very few methods are reported describing deodorization of the algae in their raw form, with no particular reference to Nannochloropsis found by the inventors of the present specification. A group of teams from a few institutions, published a study in the Journal of the Science of Food and Agriculture, 2017, vol. 97, 5123-5130, which is focused on the removal of odoriferous compounds from Arthrospira platensis, by solvent extraction and preforming trials using three different solvents—ethanol, hexane and acetone—indicating by GC-MS analysis that the ethanol extract removed the most of the odoriferous and undesirable compounds.

According to the Japanese patent application JPH06311882, algae—Crypthecodinium cohnii—are cultured to produce DHA for medical use, and odors are removed by extraction with supercritical CO₂. The efficiency of this method is strongly dependent on extraction temperature, pressure and reaction time, although it is described as having a low DHA % wt loss. It can be efficient to extract odorous compounds, only if the algae biomass has less than 20% water by weight.

A seaweed deodorization method is described in KR 101753224, according to which the seaweed is immersed in a carbonic acid buffer solution filled with carbon dioxide and high pressure (50-250 MPa) is applied. The carbon dioxide bubbles are said to selectively remove the odorous compounds from the seaweed, and with the reaction parameter adjustment—pressure, temperature, pH, CO₂ production rate, etc.—the physical properties, texture, taste and form of the product remain intact. This multi parameter adjustment however, is what renders the method unpractical and the desired product is not achieved easily. Further to this, high pressure is required, which may cause alterations in the cell integrity overall.

There is thus a need for a straightforward process to deodorize algae, and of the Nannochloropsis genus in particular. Within the biorefinery concept for sustainable manufacturing of bio-based products, such a process would rather maintain the functionality of the resulting fractions and enable their further exploitation, in order to allow for the maximum possible valorization of all production streams.

### BRIEF DESCRIPTION OF THE DRAWINGS

FIG. 1 and FIG. 2 illustrate the removal trend of the aldehydes and ketones from the Nannocloropsis oculata algal biomass according to the present invention. See also Table 1, where the respective values are depicted.

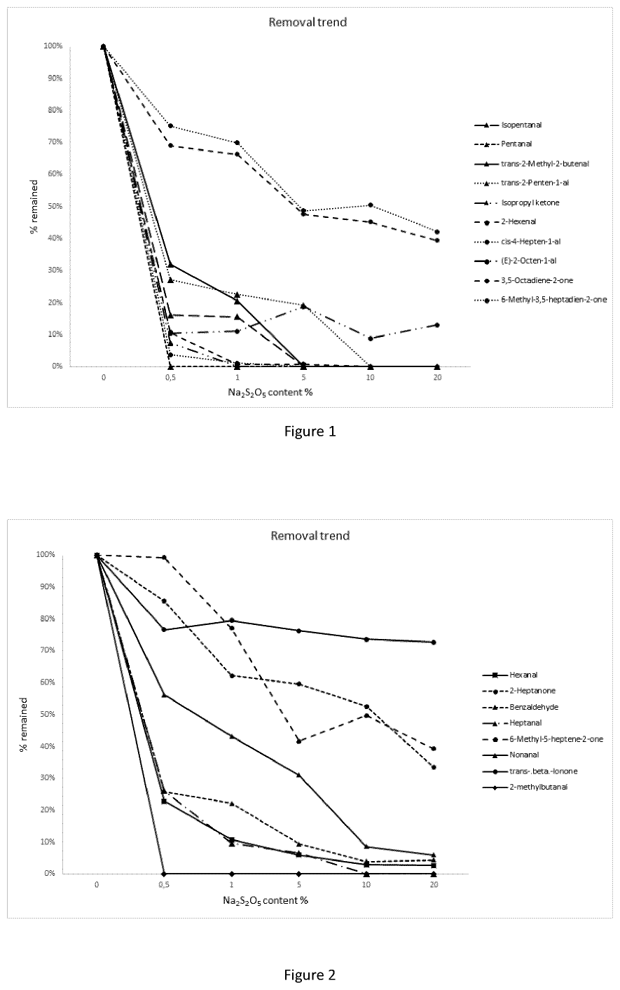

### DEFINITIONS

As used herein, “microalgae” are microscopic algae, typically found in freshwater and marine systems, living in both water and sediment. “Microalgal” may be defined in an analogous manner.

As used herein, the term “biomass” refers to carbon containing materials which result from growth of algae, but may also include material from other growing organisms. The term “microalgal biomass” and “algal biomass” are used interchangeably.

As used herein, an “adduct” is a chemical species AB, each molecular entity of which is formed by direct combination of two separate molecular entities A and B in such a way that there is change in connectivity, but no loss, of atoms within the moieties A and B.

### DETAILED DESCRIPTION

According to a first embodiment of the present invention, a process to deodorize a microalgal biomass of Nannochloropsis algae is provided, comprising

a) contacting the biomass with an adduct forming compound selected from metal sulfites, metal bisulfites, ammonium bisulfite, metal metabisulfites, SO₂ or mixtures thereof;

b) stirring or shaking;

c) collecting a solid from the suspension.

According to a second embodiment, the Nannochlropsis algae is selected from the group consisting of N. gaditana, N. granulate, N. limnetica, N. oceanica, N. oculata and N. salina. According to a preferred embodiment the Nannochlropsis algae is Nannochloropsis oculata.

The algal biomass is considered to be in dry form, when it contains less % water by weight than the harvested algal biomass. The microalgal biomass of Nannochloropsis provided in step (a) may be sun dried, oven dried, air dried, freeze dried, spray dried, or processed according to other standard food drying techniques known in the art and may contain 0 to 7% water by weight, preferably 0.5-6%, more preferably 1-5%; most preferably water content of dry biomass used in the present invention is 4% water by weight. Alternatively the algal biomass used in the present invention may be wet, with a water content of 7-99.95% by weight, preferably 7-98%, more preferably 7-85%, even more preferably 7-70% water by weight, most preferably 7-50%. In another embodiment, the wet algal biomass used in the present invention has a water content of 15-99.95% by weight, preferably 15-98%, more preferably 15-85%, even more preferably 15-70% water by weight, most preferably 15-50%.

Drying the microalgal biomass is advantageous to facilitate further processing. Drying refers to the removal of free surface moisture/water from predominantly intact biomass or the removal of surface water from a slurry of homogenized (e.g. by micronization) biomass. In some cases, drying the biomass may facilitate a more efficient microalgal oil extraction process. The deodorization method of the present invention may be performed either before or after the drying procedure on dry or wet biomass respectively.

The algal biomass is contacted with an aqueous solution of the adduct forming compound/s according to known methods to the skilled person and a suspension is formed according to step (a). A bonus to the method is that the use of organic solvent is not required.

According to step (b), the metal sulfites or SO2 react with aldehydes and/or ketones and bisulfite adducts are formed, which are subsequently removed from the algal biomass. Metal sulfites are preferably selected from sodium sulfite, potassium sulfite and lithium sulfite;

metal bisulfites are preferably selected from sodium bisulfite, potassium bisulfite and lithium bisulfite; and metal metabisulfites are preferably selected from sodium metabisulfite, potassium metabisulfite and lithium metabisulfite. Mixtures thereof may also be used. Most preferably sodium metabisulfite is used.

Bisulfite adducts formation is strongly dependent on the reactivity of the carbonyl group of the aldehydes and ketones. The inventors found that aldehydes and sterically unhindered cyclic and methyl ketones were efficiently removed from the algal biomass.

A pH adjustment step may be required in some cases, depending on the adduct forming compound used. It has been observed that in very acidic pH values, the color of the algae in the suspension changes, and this is not desired in most cases. The pH of the suspension may range between 2-12, preferably 3.5-8, more preferably 4-7, even more preferably 4-6 and most preferably 4-5.

Collecting a solid, according to step c of the present invention, is carried out by methods known to the skilled synthetic chemist for separating solids from liquids, such as filtration, or decanting the supernatant and collecting the sediment.

According to another embodiment, the process further comprises:

d) washing the solid with water and

e) optionally drying.

Washing, according to step (d), is carried out with an aqueous solution selected from water, aqueous solutions of NaCl, H₂O₂, phosphate, acetate or citric acid. Water is the preferred means.

Drying, according to step (e), may be performed by any method known to the skilled person. The inventors of the present specification, found that freeze drying is the optimal method in this case, since the organoleptic properties of the deodorized algal biomass remain intact. It was observed that by oven drying, the dry solid had a crumbly texture and a dark green-brown color, which makes it unattractive to the consumer.

Head space solid phase microextraction (HS-SPME) with gas chromatography-mass spectrometry (GC-MS) was used to analyse the deodorized algae samples reconstituted in D.M. water and measure the odorous volatile compounds content. GC-MS analysis was performed on a Shimadzu GC-2010 coupled with GCMS-Q2010^{Plus} Mass Spectrometer. Divinylbenzene/Carboxen/Polydimethylsiloxane (DVB/CAR/PDMS) SPME fibers 1 cm were used for extraction.

Table 1 shows the effect of the sodium metabisulfite concentration on the removal of the aldehydes and ketones. For the purpose of these measurements, the samples were treated with aqueous solutions of MBSF or water (control) for 10 minutes, followed by washing with DM water, and freeze drying.

TABLE 1

| | | | | | | | |
|---|---|---|---|---|---|---|---|
| | 0% | | 0.5% | 1.0% | 5.0% | 10.0% | 20.0% |
| | MBSF | | MBSF | MBSF | MBSF | MBSF | MBSF |
| Analyte | (ref. ex. 7) | | (ref. ex. 6) | (ref. ex. 5) | (ref. ex. 4) | (ref. ex. 3) | (ref. ex. 2) |
| Name | Area | Similarity¹ | Area | Area | Area | Area | Area |

Aldehydes

| | | | | | | | |
|---|---|---|---|---|---|---|---|
| Isopentanal | 1752183 | 89% | 281610 | 272720 | 60207 | ND | ND |
| 2- | 273731 | 84% | ND | ND | ND | ND | ND |
| Methylbutanal | | | | | | | |
| Pentanal | 1574983 | 90% | ND | ND | ND | ND | ND |
| trans-2- | 265148 | 93% | 84891 | 54392 | ND | ND | ND |
| Methyl-2- | | | | | | | |
| butenal | | | | | | | |
| trans-2- | 746731 | 89% | 202921 | 168191 | 143385 | ND | ND |
| Penten-1-al | | | | | | | |
| Hexanal | 10681018 | 97% | 2433369 | 1152529 | 636175 | 315941 | 290714 |
| 2-Hexenal | 1132379 | 91% | 120636 | 6810 | 8707 | ND | ND |
| cis-4-Hepten- | 1492530 | 92% | 55678 | ND | ND | ND | ND |
| 1-al | | | | | | | |
| Heptanal | 3635617 | 97% | 946459 | 350191 | 234851 | ND | ND |
| Benzaldehyde | 1878233 | 96% | 486352 | 415371 | 176306 | 73539 | 81960 |
| (E)-2-Octen- | 323309 | 94% | 33573 | 35554 | 60452 | 28597 | 42087 |
| 1-al | | | | | | | |
| Nonanal | 833685 | 95% | 468292 | 359572 | 258248 | 71750 | 49862 |

Ketones

| | | | | | | | |
|---|---|---|---|---|---|---|---|
| Isopropyl | 1383025 | 93% | 103264 | ND | ND | ND | ND |
| ketone | | | | | | | |
| 6-Methyl-5- | 13430122 | 93% | 13320616 | 10350409 | 5585488 | 6665486 | 5272939 |
| heptene-2- | | | | | | | |
| one | | | | | | | |
| 3,5-Octadiene-2- | 1463160 | 91% | 1008530 | 970487 | 696176 | 659840 | 575853 |
| one | | | | | | | |
| 6-Methyl- | 385529 | 89% | 289636 | 269243 | 187707 | 194345 | 162380 |
| 3,5-heptadien-2- | | | | | | | |
| one | | | | | | | |
| 2-Heptanone | 481184 | 84% | 412050 | 299070 | 287153 | 252852 | 161082 |
| trans-beta- | 3248251 | 94% | 2490068 | 2584660 | 2479652 | 2389275 | 2363725 |
| Ionone | | | | | | | |

¹Similarity of the mass-to-charge ratio (m/z) to ions of known m/z. One of the commonly used approaches for annotation of mass spectra is the similarity search in a database of theoretical spectra generated from a database of substances.

The content of the odorous compounds in the deodorized algae biomass is either reduced to non-detectable levels, either significantly reduced, and this ability is seen to improve by increase of the sodium metabisulfite concentration. The latter may range from 0.5% to 30%, preferably may be 0.5%, 1%, 5%, 10%, 15%, 15%, 20%, 25% or 30% by weight, and most preferably 20% by weight.

Another major advantage of the deodorization method described herein, is that no significant EPA loss was observed. Minimal loss of other fatty acids present in the algal biomass was also found.

The process is further non-destructive and reversible, maintaining the functionality of the products. The formed adducts may be further treated to form the compounds that were initially removed from the algal biomass and which may have valuable properties. The simple, yet very efficient method to deodorize Nannochloropsis algae biomass, which selectively removes the odorous compounds, leaving the nutritional components fairly intact is illustrated by way of examples.

### EXAMPLES

The Nannochloropsis used in the examples of the present invention, were spray dried after harvesting, and contained up to 7% water by weight.

### Example 1

A 250 mL round bottom flask equipped with a magnet stirring bar is charged with 20 g N. oculata powder followed by 100 mL sodium metabisulfite solution 20% w/v. The suspension is stirred under inert atmosphere for 2.5 hours at ambient temperature and then, the solid is collected by filtration under reduced pressure. The filter cake is spray-washed with 2×100 mL D.M. water, suck dried for 30 minutes and then dried under vacuum in an oven for 2 hours at 45 ° C. to afford 17.6 g of dry powder.

### Example 2

A 250 mL round bottom flask equipped with a magnet stirring bar is charged with 20 g N. oculata powder followed by 100 mL sodium metabisulfite solution 20% w/v. The suspension is stirred under inert atmosphere for 2.5 hours at ambient temperature and then, the solid is collected by filtration under reduced pressure. The filter cake is spray-washed with 2×100 mL D.M. water, suck dried for 30 minutes and then freeze-dried for 18 hours to afford 17.2 g of dry powder.

### Example 3

A 250 mL round bottom flask equipped with a magnet stirring bar is charged with 20 g N. oculata powder followed by 100 mL sodium metabisulfite solution 10% w/v. The suspension is stirred under inert atmosphere for 2.5 hours at ambient temperature and then, the solid is collected by filtration under reduced pressure. The filter cake is spray-washed with 2×100 mL D.M. water, suck dried for 30 minutes and then freeze-dried for 18 hours to afford 17.4 g of dry powder.

### Example 4

A 250 mL round bottom flask equipped with a magnet stirring bar is charged with 20 g N. oculata powder followed by 100 mL sodium metabisulfite solution 5% w/v. The suspension is stirred under inert atmosphere for 2.5 hours at ambient temperature and then, the solid is collected by filtration under reduced pressure. The filter cake is spray-washed with 2×100 mL D.M. water, suck dried for 30 minutes and then freeze-dried for 18 hours to afford 17.0 g of dry powder.

### Example 5

A 250 mL round bottom flask equipped with a magnet stirring bar is charged with 20 g N. oculata powder followed by 100 mL sodium metabisulfite solution 1% w/v. The suspension is stirred under inert atmosphere for 2.5 hours at ambient temperature and then, the solid is collected by filtration under reduced pressure. The filter cake is spray-washed with 2×100 mL D.M. water, suck dried for 30 minutes and then freeze-dried for 18 hours to afford 16.9 g of dry powder.

### Example 6

A 250 mL round bottom flask equipped with a magnet stirring bar is charged with 20 g N. oculata powder followed by 100 mL sodium metabisulfite solution 0.5% w/v. The suspension is stirred under inert atmosphere for 2.5 hours at ambient temperature and then, the solid is collected by filtration under reduced pressure. The filter cake is spray-washed with 2×100 mL D.M. water, suck dried for 30 minutes and then freeze-dried for 18 hours to afford 17.4 g of dry powder.

### Example 7

A 250 mL round bottom flask equipped with a magnet stirring bar is charged with 20 g N. oculata powder followed by 100 mL D.M. water. The suspension is stirred under inert atmosphere for 2.5 hours at ambient temperature and then, the solid is collected by filtration under reduced pressure. The filter cake is spray-washed with 2×100 mL D.M. water, suck dried for 30 minutes and then freeze-dried for 18 hours to afford 17.3 g of dry powder.

### Example 8

A 250 mL round bottom flask equipped with a magnet stirring bar is charged with 20 g N. oculata powder followed by 100 mL sodium bisulfite solution 20% w/v at pH 4—5 adjusted with sodium phosphate dibasic. The suspension is stirred under inert atmosphere for 2.5 hours at ambient temperature and then, the solid is collected by filtration under reduced pressure. The filter cake is spray-washed with 2×100 mL D.M. water, suck dried for 30 minutes and then freeze-dried for 18 hours to afford 17.0 g of dry powder.

### Example 9

A 250 mL round bottom flask equipped with a magnet stirring bar is charged with 20 g N. oculata powder followed by 100 mL sodium bisulfite solution 5% w/v at pH 4—5 adjusted with sodium phosphate dibasic. The suspension is stirred under inert atmosphere for 2.5 hours at ambient temperature and then, the solid is collected by filtration under reduced pressure. The filter cake is spray-washed with 2×100 mL D.M. water, suck dried for 30 minutes and then freeze-dried for 18 hours to afford 17.2 g of dry powder.

1. A process for deodorizing a Nannochloropsis algal biomass, comprising a) contacting the biomass with an adduct forming compound selected from metal sulfites, metal bisulfites, ammonium bisulfite, metal metabisulfites, SO₂, or mixtures thereof, b) stirring or shaking, and c) collecting a solid from the suspension.

2. A process according to claim 1, wherein the Nannochloropsis is selected from the group consisting of N. gaditana, N. granulate, N. limnetica, N. oceanica, N. oculata and N. salina.

3. A process according to claim 2, wherein the Nannochloropsis is Nannochloropsis oculata.

4. The process according to any preceding claim, wherein the adduct forming compound in step (a) is either a metal sulfite selected from sodium, potassium sulfite and mixtures thereof, a metal bisulfite selected from sodium bisulfite, potassium bisulfite, and mixtures thereof, or a metal metabisulfite selected from sodium and potassium metabisulfite and mixtures thereof.

5. The process according to any preceding claim, wherein step (b) is performed for about 30 minutes to about 6 hours.

6. The process according to any preceding claim, wherein step (c) is performed by filtration or by decanting the supernatant and collecting the sediment.

7. The process according to any preceding claim, further comprising d) washing the solid with an aqueous solution and e) optionally drying.

8. The process according to claim 7, wherein the solid is washed with an aqueous solution selected from water, NaCl, H₂O₂, phosphate or citric acid solution.

9. The process of claim 7, wherein the solid is washed with water.

10. The process according to any preceding claim, wherein the Nannochloropsis algal biomass is in dry form.

11. The process according to any preceding claim, wherein the Nannochloropsis algal biomass is in sun dried, oven dried, air dried, spray dried or freeze dried form.

12. The process according to claims 1-9, wherein the Nannochloropsis algal biomass is in wet form.
